(19) United States
(12) Patent Application Publication
(10) Pub. No.: US 2007/0149977 A1
(43) Pub. Date: Jun. 28, 2007

(54) Surgical component positioner

(75) Inventor: Jackson R. Heavener, Warsaw, IN (US)

Correspondence Address: John F. Hoffman, Esq.;BAKER & DANIELS LLP, Suite 800, 111 East Wayne Street, Fort Wayne, IN, 46802, US

(73) Assignee: Zimmer Technology, Inc.

(21) Appl. No.: 11/287,839

(22) Filed: Nov. 28, 2005

(51) Int. Cl. A61F 5/00

(52) U.S. Cl. 606/87

(57) ABSTRACT

A surgical component positioner positions a surgical component at a surgical site. The surgical component positioner includes a body, a component link adjustably mounted to the body with multiple degrees of freedom relative to the body; and an adjustable locking mechanism operably connected to the component link. The locking mechanism includes at least three operation states. In a first state the component link is freely positionable by a user relative to the body. In a second state component link is fluidly positionable such that it is readily positionable by a user relative to the body and self supporting such that it will maintain its position when released. In a third state the component link position is locked relative to the body. In one embodiment, the surgical component includes a cut guide linked to the body such as for guiding a cutter for cutting a tibial bone or a femoral bone during knee replacement surgery.

### FIELD OF THE INVENTION

The present invention relates to a surgical instrument positioner for supporting a surgical component at a desired location relative to a surgical site.

### BACKGROUND

Degenerative and/or traumatic damage to skeletal joints or other locations within a patient's body may require surgical intervention. During such surgical intervention, it is often necessary to position and/or support a surgical component at a desired location relative to the surgical site. Surgical components may include cutting instruments, cutting guides, trial implants, implants, and/or other surgical components. For example, damage to the articular cartilage of a skeletal joint can result in pain and restricted motion. Prosthetic joint replacement is frequently utilized to alleviate the pain and restore joint function. In this procedure, the damaged parts of the joint are cut away and replaced with prosthetic components. Typically a resection guide is used to guide a cutter such as a saw blade or bur to cut a desired portion of the bone to prepare a seating surface for a prosthetic component. The resection guide must be carefully positioned to guide the cut at the appropriate location. The prosthetic component must then be carefully positioned in the prepared space.

Many surgical procedures are now performed with the aid of surgical navigation systems in which sensors detect tracking elements attached in known relationship to an object in the surgical suite such as a surgical instrument, implant, or patient body part. The sensor information is fed to a computer that then triangulates the three dimensional position of the tracking elements within the surgical navigation system coordinate system. Thus, the computer can resolve the position and orientation of the object and display the position and orientation for surgeon guidance. For example, the position and orientation can be shown superimposed on an image of the patient's anatomy obtained via X-ray, CT scan, ultrasound, or other imaging technology.

### SUMMARY

The present invention provides a surgical component positioner for positioning a surgical component at a surgical site

In one aspect of the invention the surgical component positioner includes a body and a component link adjustably mounted to the body with multiple degrees of freedom relative to the body. An adjustable locking mechanism has at least three operation states including: a first state in which the link is freely positionable by a user relative to the body, a second state in which the link is fluidly positionable such that it is readily positionable by a user relative to the body and self supporting such that it will maintain its position when released, and a third state in which the link position is locked relative to the body.

In another aspect of the invention, a cut guide positioner for knee surgery includes a body and a cut guide linked to the body with multiple degrees of rotational freedom relative to the body. An adjustable locking mechanism has at least three operation states including: a first state in which the cut guide is freely positionable by a user relative to the body in the multiple degrees of rotational freedom, a second state in which the cut guide is fluidly positionable by a user relative to the body in the multiple degrees of freedom such that in the second state the cut guide is readily positionable by a user and is self supporting such that it will maintain its position when released, and a third state in which the cut guide position is locked relative to the body.

In another aspect of the invention, a method for positioning a surgical component adjacent a surgical site includes providing a surgical component positioner having a body, a surgical component linked to the body by a ball joint with multiple degrees of freedom relative to the body, and an adjustable locking mechanism; adjusting a first angular position of the surgical component relative to the body by angling the ball joint with the locking mechanism in an initial position; adjusting a second angular position of the surgical component relative to the body by angling the ball joint with the locking mechanism in the initial position; and moving the locking mechanism to a locked position to lock the first and second angular positions of the surgical component.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is an exploded perspective view of an exemplary surgical component positioner according to the present invention;

FIG. 2 is a cross sectional view of the positioner of FIG. 1 attached to a bone; and

FIG. 3 is a cross sectional view of the cam locking mechanism of the positioner of FIG. 1.

### DESCRIPTION OF THE ILLUSTRATIVE EXAMPLES

Embodiments of a surgical component positioner include a body and a component link adjustably mounted to the body. The component link may be formed on the surgical component or it may be a separate element interposed between the surgical component positioner and the surgical component. The component link may be mounted with one or more degrees of freedom relative to the body. For example, the component link may be mounted to the body with three rotational degrees of freedom. For example, the component link may include a hemispherical ball that is mounted in a hemispherical socket on the body to provide three rotational degrees of freedom. The link may be freely rotatable with respect to all degrees of freedom relative to the body. Alternatively, the link may be restrained in one or more degrees of freedom such that it is fluidly adjustable relative to the body but will maintain its orientation absent a positioning force from a user. Alternatively, the link may be locked with respect to one or more degrees of freedom such that the link is locked in place relative to the body. The component positioner may include an adjustable locking mechanism operable to release the link for free positioning, partially restrain the link for fluid, but self supporting, positioning, and/or lock the link rigidly relative to the body. For example, the locking mechanism may comprise a cam lock having three positions. The locking mechanism may further include a plunger activated by a cam to impinge on a ball head formed on the link to vary the adjustability of the link from free, to restrained, to locked.

The positioner body may be mounted directly to a surgical site. For example, the body may be pinned, screwed, dovetail engaged, and/or otherwise mounted to a bone adjacent a skeletal joint. Alternatively, the positioner may include a linear adjustment mechanism for translating the body and attached component relative to a surgical site. For example, the body may be mounted on a support for linear translation. The body may slide along the support in response to being pushed by a user. Alternatively, the body may be positionable by turning a knob to move the body along the support. For example, the linear adjustment mechanism may include a rack and pinion mounting. For example, the support may include one or more linear racks along which the body translates and the body may include one or more corresponding pinions connected to one or more knobs. Upon rotating the knob, the pinion engages the rack and forces the body to translate along the rack. The body may be moveable freely along the support to a desired position. Alternatively, the body may be restrained to move fluidly along the support but maintain its position on the support absent a positioning force from a user. Alternatively, the body may be locked in position relative to the support. The component positioner may include a locking mechanism operable to release the body for free positioning relative to the support, partially restrain the body for fluid, but self supporting, positioning relative to the support, and/or lock the body rigidly relative to the support. For example, the locking mechanism may comprise one or more tensioning screws operable to increase friction in the linear adjustment mechanism from little or no friction for free movement to moderate friction for fluid movement to high friction for locking. For example, a tension screw may be mounted to the body with a spring and ball between the screw and the support. Upon tightening the tension screw, the screw may compress the spring and press the ball into engagement with the support such that progressive tightening of the screw progressively restrains motion of the body relative to the support. The screw may be arranged to fully compress the spring at one position of screw travel to facilitate high engagement forces to lock the linear adjustment mechanism to permit another degree of translational freedom and consequently more adjustability . . .

The linear adjustment mechanism may be mounted directly to a surgical site. For example, the support may be screwed, pinned, dovetail engaged, and/or otherwise mounted to a bone adjacent a skeletal joint. Alternatively, the positioner may include a base for mounting the linear adjustment mechanism. The base may include holes, slots, notches, and/or other suitable features for receiving fixation members to mount the base. The base may also include projecting spikes, ribs, threaded posts, and/or other suitable features for engaging the surgical site. For example, the base may include a spike for insertion into a bone adjacent a skeletal joint. The base may connect to the linear adjustment mechanism by unitary fabrication, welding, screwing, dovetail engagement, and/or other suitable connection mechanism. The base may be fixed relative to the linear adjustment mechanism or it may further incorporate one or more additional degrees of adjustment freedom. For example, the base may be mounted to the linear adjustment mechanism by way of a sliding dovetail. The sliding dovetail may be oriented for motion perpendicular to the motion of the linear adjustment mechanism.

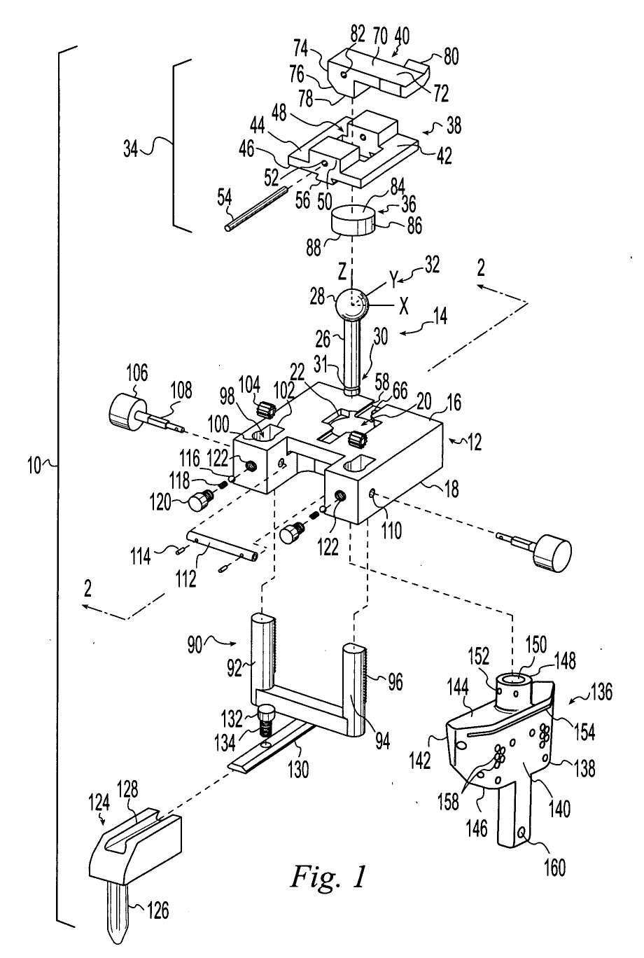

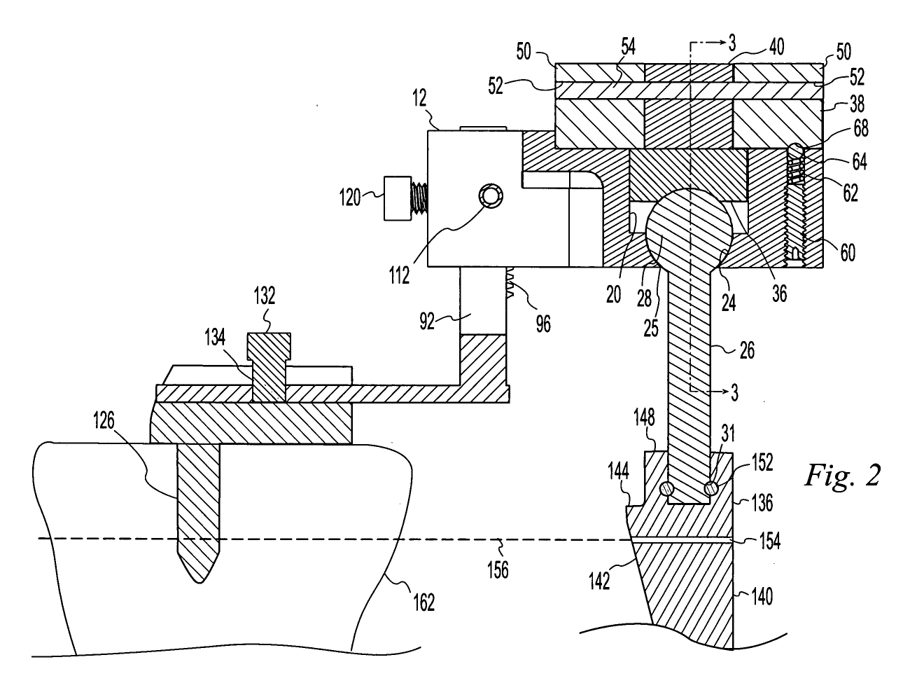

FIGS. 1 and 2 depict an exemplary surgical component positioner 10 having a body 12 and a component attachment link 14. The body 12 is in the form of a rectangular prism having a top surface 16, a bottom surface 18, and four side walls. A cylindrical cavity 20 extends into the body 12 from the top surface 16 to define an opening 22 at the top surface. A hemispherical seat 24 (FIG. 2) is formed opposite the cavity opening 22 and includes a central through bore 25 communicating between the cavity 20 and the bottom surface 18. The link 14 includes an elongated shaft 26 with a hemispherical head 28 at one end and a component attachment portion 30 including an annular groove 31 at the opposite end. The link shaft 26 inserts through the bore 24 and the hemispherical head 28 fits into the hemispherical seat 24 such that the component attachment portion 30 extends below the bottom surface 18 of the body 12. The ball and socket joint between the link 14 and the body 12 permits three degrees of rotational freedom of the link 14 relative to the body 12. This rotation freedom can be characterized as rotation about each of the X, Y, and Z-axes of the coordinate system 32 shown in FIG. 1.

An adjustable locking mechanism 34 includes a plunger 36, a cam support 38, and a cam lock 40. The cam support 38 includes a body 42 in the form of a rectangular prism having a top surface 44, a bottom surface 46, and a peripheral side wall. A central opening 48 communicates from the top surface 44 to the bottom surface 46. A pair of riser blocks 50 project upwardly from the top surface 44 on opposite sides of the opening 48. Aligned bores 52 receive a pivot pin 54 for mounting the cam lock 40. Male dovetail slide segments 56 project from the bottom surface 46 to slidingly engage a dovetail slot 58 formed in the positioner body 12. The dovetail slot 58 is centered over the cavity opening 22 and extends on either side of the opening 22. The dovetail slot 58 opens outwardly at the sidewall. The cam support 38 is mounted on the positioner body 12 by sliding the dovetail slide 56 into the dovetail slot 58. Providing the cam support 38 as a separate and removable piece facilitates maintenance and cleaning of the surgical component positioner 10 and facilitates exchanging the component attachment link 14. Alternatively, the cam support may be formed as a unitary part of the positioner body 12.

The cam support 38 is retained by a resilient ball mechanism mounted in the positioner body 12 (FIG. 2). The mechanism includes a mounting/adjustment screw 60, a spring 62, and a ball 64. The spring 62 biases the ball 64 upwardly through an opening 66 communicating with the dovetail slot 58. The opening 66 is smaller than the diameter of the ball 64 so that the ball is prevented from passing completely through the opening 66. The dovetail slide 56 includes a hemispherical depression 68 for the ball 64 to seat in. As the dovetail slide 56 is engaged with the dovetail slot 58, the dovetail slide 56 presses the ball 64 downwardly against the spring 62 and the dovetail slide 56 slides across the ball. As the depression 68 aligns with the ball 64, the spring 62 biases the ball upwardly into engagement with the depression 68. To remove the cam support 38, increased sliding pressure is required to overcome the spring tension and force the ball 64 downwardly. The required removal force may be adjusted by tightening or loosening the screw 60 to vary the preload tension of the spring 62.

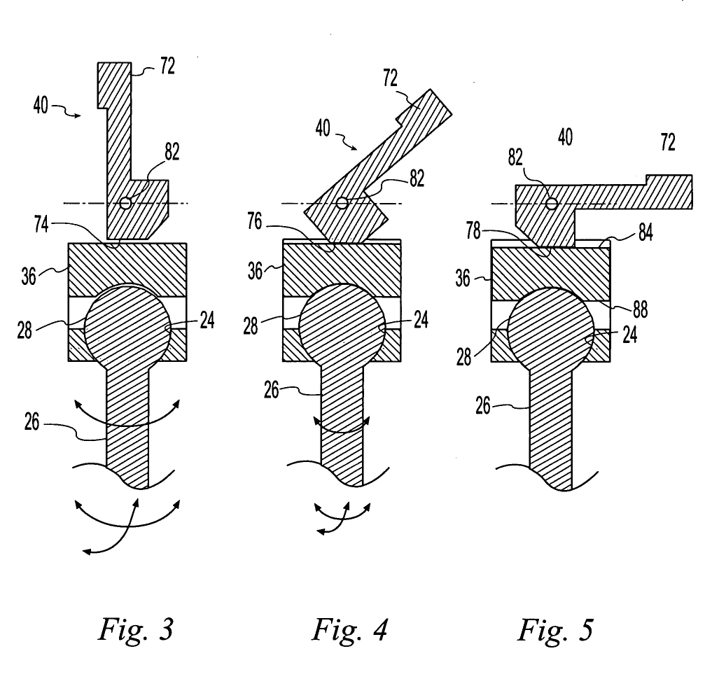

The cam lock 40 includes an elongated body 70 having a handle 72 at a first end and three cam faces 74, 76, 78 at the opposite, second end. The handle 72 includes an upturned lip 80 to make it easier to grip the handle and lift it from the top surface of the cam support 38. The second end includes a through bore 82 for receiving the pivot pin 54. Each of the three cam faces 74, 76, 78 is spaced a different distance from the bore 82 than each of the other faces. The cam faces 74, 76, 78 may meet at sharp vertices as shown or they may have radii between them to ease operation of the cam lock 40. As the cam lock 40 is rotated about the pivot pin 54, each of the cam faces 74, 76, 78 can be positioned parallel to the top of the cam support 38 such that each cam face 74, 76, 78 will project downwardly from the pivot pin a different distance. This can best be seen in FIGS. 3-5 and will be discussed in more detail with reference to the operation of the surgical component positioner.

The plunger 36 is in the form of a cylindrical disk having a top surface 84, a side wall 86, and a bottom surface 88. The diameter of the plunger 36 is sized for a sliding fit within the cylindrical cavity 20 in the positioner body 12. The bottom surface 88 of the plunger 36 is formed into a hemispherical seat to engage the top of the ball head 28 of the link 14. A separate plunger 36 permits the use of separate materials such as metal for the cam lock for strength and a polymer for the plunger 36 for smooth operation. The plunger material may also be selected for high friction coefficient relative to the link 14, for compressibility to improve it's grip on the link 14, and/or for other properties. Alternatively, the plunger may be omitted and the cam lock 40 may engage the link 14 directly.

The positioner body 12 is mounted on a support 90 by way of a linear adjustment mechanism for translating the body 12 relative to a surgical site. The support 90 includes a pair of upwardly extending arms 92. Each arm has a “D” shaped cross section with the curved side 94 being smooth and the flat side 96 being formed into a toothed rack. The positioner body 12 includes a pair of openings 98 extending from the top surface 16 to the bottom surface 18. A first half 100 of each opening 98 is shaped to receive the smooth side of one of the arms 92 for up and down translation. A second half 102 of each opening 98 is rectangular and offset from the first half 100. A toothed pinion 104 is mounted in the second half of each opening 98. A pair of adjustment knobs 106 include elongated shafts 108. The shaft 108 of each knob 106 is mounted for rotation in aligned through bores 110 intersecting the second half 102 of each opening 98. The pinions 104 are rotationally keyed to the shafts 108. A connector tube 112 is positioned between and receives a portion of each of the knob shafts 108. The connector tube is rotationally locked to each shaft by a cross pin 114. Thus, the knobs 106 are rotationally linked such that when either of the knobs 106 is turned both knobs and consequently both pinions turn. The pinions 104 are spaced from the first half 100 of each opening such that when the arms 92 of the support 90 are engaged with the first half 100 of the opening, the pinion teeth engage the rack teeth. By turning either one, or both, of the adjustment knobs the pinions 104 will move along the racks and thus cause the positioner body 12 to move up and down on the support 90.

A support locking mechanism includes a ball 116, a spring 118, and a tension screw 120 mounted in a bore 122 in the positioner body 12 communicating with each of the openings 98. The bore 122 is aligned with the opening 98 so that the ball 116 is biased by the spring against the curved, smooth side of each arm 92. Tightening the tension screw 120 increases the pressure by which the ball 116 is pressed against the arm 92 and thereby increases the force required to adjust the positioner body 12 up and down on the support 90. Progressive tightening of the tension screws 120 eventually increases the adjustment force to a level at which the relative position of the body 12 and support 90 are essentially locked. If the tension screws 120 are tightened until the spring 118 is fully compressed, the locking force will rise rapidly with increased tightening of the tension screws 120.

The exemplary surgical component positioner 10 is provided with a base 124 for mounting the positioner 10 to a bone. The base 124 includes a projecting spike 126 for insertion into a bone. The base 124 further includes a dovetail slot 128 formed opposite the spike 126. The dovetail slot 128 slidingly receives a dovetail slide 130 projecting from the support 90 to permit a single degree of linear translation of the support 90 relative to the base 124. A knob132 is threadingly engaged with the dovetail slide 130 and can be tightened so that the shaft 134 of the knob 132 presses against the bottom of the dovetail slot 128 and locks the position of the dovetail slide 130 within the dovetail slot 128.

An exemplary surgical component is depicted in the form of a cut guide 136. The cut guide 136 is positioned adjacent to a bone and used to guide a cutter to cut the bone. The exemplary cut guide includes a body 138 having a front surface 140, a back surface 142, a top 144, and a bottom 146. A spigot 148 extends upwardly from the top 144 of the cut guide 136 and defines an internal bore 150 sized to receive the component attachment portion 30 of the link 14. A pair of resilient wires 152 are mounted transversely in the bore 150 to engage the annular groove 31 of the component attachment portion 30 of the link 14 in snap-fitting relationship. The bore 150 and attachment portion 30 may be cylindrical to permit a single degree of rotational freedom between the link 14 and cut guide 136 as shown. Alternatively, the bore 150 and attachment portion 30 may include flats or other rotational keys to prevent rotation between them. The cut guide 136 includes a saw guide slot 154 defining a cut plane 156 (FIG. 2). The cut guide 136 further includes fixation holes 158 extending from the front surface 140 to the back surface 142. The holes 158 optionally receive fixation elements (not illustrated) such as pins, wires, screws, and/or other suitable fixation elements to secure the cut guide 136 to a bone prior to the cut guide 136 being used to guide a cutter (not illustrated).

The surgical component positioner 10 may optionally be used with a surgical navigation system to aid in positioning the surgical component. A surgical navigation system includes one or more tracking elements that are detectable electromagnetically, acoustically, optically, and/or by other suitable detection means. The tracking element may be active or passive. For example, tracking elements may include reflective spheres, light emitting diodes, gyroscopic sensors, electromagnetic emitters, electromagnetic receivers, and/or other suitable tracking elements. The tracking element (or elements) may be positioned on the component attachment link 14 to indicate the position of the link 14. The position of the surgical component may then be resolved by the surgical navigation system from a predetermined relationship between the link and the component. Preferably, the tracking element is positioned on the surgical component to directly indicate the position of the component within the surgical navigation coordinate system. By directly navigating the surgical component, positional errors due to tolerance stack-up in the connection between the link 14 and the component are eliminated. For example, in the exemplary cut guide 136, it is desirable to carefully position the cut plane 156. Therefore, a tracking element 160 is attached to the cut guide 136 in known relationship to the saw guide slot 154. During use, the surgical component positioner 10 provides a stable base for adjusting the cut guide 136 while it is navigated into position.

For example, the tracking element 160 may be in the form of an electromagnetic coil. The tracking element 160 is detectable by the surgical navigation system such that the three dimensional position and orientation of the tracking element can be related to the surgical navigation coordinate system. For example, the surgical navigation system may include multiple sensors at known locations that receive signals from the tracking element 160 and feed the information to a computer. The computer may then triangulate the three dimensional position of the tracking element 160 within the surgical navigation coordinate system. The surgical navigation system may then determine the position and orientation of the saw guide slot 154 by detecting the position and orientation of the tracking element 160 and resolving the position and orientation of the saw guide slot 154 from the known relationship between the tracking element 160 and the saw guide slot 154. The surgical navigation system may then determine the location of the cut plane 156 relative to the patient's anatomy to guide the surgeon in positioning the cut guide 136. For example, the surgical navigation system may superimpose the computed cut plane 156 onto an image of the patient's anatomy so that the surgeon may visualize the cut plane 156 location. In another example, the desired cut location may be indicated on a computer model of the patient's anatomy prior to surgery and the surgical navigation may indicate by way of optical and/or auditory feedback when the cut plane 156 is aligned with the predetermined cut location. A surgical navigation system may be used in any other suitable manner to aid the user in aligning a surgical component relative to a surgical site.

The surgical component positioner 10 is shown in use to position a tibial cut guide adjacent a proximal portion of a tibia in total knee replacement surgery to establish the varus/valgus angle, anterior/posterior slope, and vertical height of the tibial component. However, the surgical component positioner 10 is useful for positioning a tibial cut guide for unicondylar knee replacement surgery, for positioning a femoral cut guide, and for positioning any surgical component adjacent to any surgical site. For example, in a femoral cut guide application, the surgical component positioner may be adjusted to set the varus/valgus angle and the internal/external rotation angle for a cutting plane. The vertical linear translation adjustment may used to adjust the vertical position of the femoral cutting plane to establish the amount of femoral bone to be removed.

The surgical component positioner is particularly useful in minimally invasive surgical procedures to adjust and hold the position of a surgical component where it may otherwise be difficult due to confined spaces or poor visualization. The optional use of a surgical navigation system with the surgical component positioner further enhances its ease of use and is especially helpful during minimally invasive procedures where the surgical navigation system can guide aligning the surgical component with anatomical features not visible to the surgeon.

In use, the exemplary surgical component positioner 10 is assembled by inserting the attachment link 14 into the cavity 20 so that the hemispherical head 28 engages the hemispherical seat 24. The plunger 36 is inserted into the cavity 20 so that the hemispherical seat of the bottom surface 88 engages the hemispherical head 28. The cam support 38 is assembled to the body 12 by sliding the male dovetail slide segments 56 into the dovetail slot 58 until the ball 64 snaps into the depression 68 to retain the cam support 38. The cut guide 136 is connected to the attachment link 14 by inserting the attachment portion 30 of the link 14 into the bore 150 in the cut guide 136 until the wires 152 snap into the annular groove 31.

The base 124 is attached to the tibia 162 by driving the spike 126 into the proximal portion of the bone. In a tibial knee application, as shown, the rotational degrees of freedom of the link 14 permit adjusting the anterior/posterior slope and the varus/valgus angle of the cut plane 156. The anterior/posterior slope is set by rotation about the X-axis and the varus/valgus angle is set by rotation about the Y-axis. With the cam lock 72 fully raised (FIG. 3), the first cam face 74 overlies the plunger and it exerts no compressive force on the plunger 36. In this first position, the attachment link 14 is freely positionable. With the cam lock 72 flipped to the midway position (FIG. 4), the second cam face 76 overlies the plunger. The second cam face 76 extends further below the pivot bore 82 and exerts a relatively moderate compressive force on the plunger 36. In this second position, the attachment link 14 is fluidly positionable such that it can be rotated about the X, Y, and Z-axes but the locking mechanism 34 will maintain the attachment link and thus the surgical component's position when they are released by the user. With the cam lock 72 flipped to the fully locked position (FIG. 5), the third cam face 78 overlies the plunger. The third cam face 78 extends further below the pivot bore 82 and exerts a relatively large compressive force on the plunger 36. In this third position, the large compressive forces between the hemispherical head 28, the plunger 36, and the seat 24 essentially lock the link 14 relative to the body 12.

The adjustment knobs 106 can be rotated to move the body 12 up and down on the support 90 to set the vertical position of the surgical component. Depending on the surgical approach and patient positioning, the user may only have convenient access to one of the knobs 106. However, because the knobs 106 are linked by connector tube 112, turning one knob will cause both pinions 104 to turn against both racks 96 and permit smooth, non-binding operation. In the exemplary case of a tibial cut guide 136, the knobs 106 can be rotated to set the vertical position of the cut plane 156 which determines how much of the proximal tibial bone will be resected. The ease and/or fluidity of the vertical adjustment can be adjusted by tightening or loosening the tension screws 120. The tension screws 120 can be tightened further to lock the vertical adjustment.

The horizontal position of the assembly can be adjusted by sliding the dovetail slide 130 of the support 90 within the dovetail slot 128 of the base 124. The force required to slide the support 90 may be increased by tightening the knob 132. The horizontal position may be locked by further tightening the knob 132. In the exemplary case of a tibial cut guide 136, sliding the assembly on the base allows the cut guide 136 to be positioned close to the bone.

The exemplary cut guide 136 connects to the attachment link 14 for free rotation of the attachment portion 30 within the bore 150. The axis of this rotation is perpendicular to the cut plane 156 so it does not change the cut angle or depth of cut of the plane relative to the bone. Therefore, the cut guide 136 can be rotated to fit close against the bone and/or to facilitate directing a saw blade at a desired part of the bone within the cut plane. Once all adjustments have been made, the cut guide 136 is optionally anchored to the bone by inserting fixation members through the fixation holes 158. The surgical component positioner may optionally be removed during the actual cutting of the bone.

Surgical navigation may be used with the surgical component positioner. For example, a surgical navigation system may be activated to track the position of the tracking element 160 and give feedback to the user indicating the position of the surgical component relative to the surgical site. In the exemplary cut guide, the system can feed back the position of the cut plane 156 relative to the bone. The cam lock 72, tension screws 120, and knob 132 may be left loose initially to allow quick rough positioning of the cut guide 136. Once the positioning is close, the cam lock 72 is advantageously moved to its second position to facilitate fine adjustments without risk of losing the adjustment if the user momentarily releases his grip. Similarly, the tension screws 120 and 132 may be tightened to yield a smoother, more controlled, and/or more stable adjustment. The surgical component positioner will hold the position of the cut guide 136 while the user diverts his attention to other matters such as adjusting other instruments, adjusting lighting, repositioning himself relative to the patient, evaluating the surgical navigation system feedback, extending a saw blade through the saw guide slot 154 to check alignment, and/or otherwise diverting his attention from the cut guide 136 position. Once the user is satisfied with the cut guide 136 position, he can advantageously lock the position by flipping the cam lock 72 to the locked position and tightening the tension screws 120 and knob 132. The cut guide 136 is optionally anchored to the bone by inserting fixation members through the fixation holes 158.

Although examples of a surgical component positioner and its use have been described and illustrated in detail, it is to be understood that the same is intended by way of illustration and example only and is not to be taken by way of limitation. The invention has been illustrated in use to position a tibial cut guide adjacent the proximal tibia in a knee replacement procedure. However, the surgical component positioner may be used at other locations within a patient's body to position other surgical components. Accordingly, variations in and modifications to the surgical component positioner and its use will be apparent to those of ordinary skill in the art, and the following claims are intended to cover all such modifications and equivalents.

What is claimed is:

1. A surgical component positioner for positioning a surgical component at a surgical site, the surgical component positioner comprising:
a body;
a component link adjustably mounted to the body with multiple degrees of freedom relative to the body; and
an adjustable locking mechanism operably connected to the component link, the locking mechanism having at least three operation states, a first state in which the component link is freely positionable by a user relative to the body, a second state in which the component link is fluidly positionable such that it is readily positionable by a user relative to the body and self supporting such that it will maintain its position when released, and a third state in which the component link position is locked relative to the body.

2. The surgical component positioner of claim 1 wherein the locking mechanism comprises a cam lock having three distinct cam faces alternatively engageable to produce the three operation states.

3. The surgical component positioner of claim 1 wherein the component link comprises a hemispherical head and the body includes a hemispherical seat receiving the head for three rotational degrees of freedom, the locking mechanism comprising a plunger engageable with the head, the cam lock being operable to engage each cam face with the plunger to provide three distinct levels of engagement of the plunger with the head to provide three distinct levels of restraint to motion of the head within the seat.

4. The surgical component positioner of claim 3 further comprising a linear adjustment mechanism including a support engaging the body for linear translation of the body relative to the support to permit linear translation of the body and component link relative to a surgical site.

5. The surgical component positioner of claim 4 wherein the linear adjustment mechanism further comprises an adjustment knob, the knob being operable to translate the body relative to the support.

6. The surgical component positioner of claim 5 wherein the linear adjustment mechanism comprises a rack mounted to the support and a pinion coupled to the knob such that rotating the knob causes the pinion to rotate in rack-and-pinion engagement with the rack.

7. The surgical component positioner of claim 6 wherein the linear adjustment mechanism further comprises a tensioning mechanism having a least two operation states, a first state in which the body is readily positionable by a user relative to the support and a second state in which the component link position is locked relative to the body.

8. The surgical component positioner of claim 7 wherein the adjustable tensioning mechanism has at least three operation states, the body being freely positionable relative to the support in the first state and the body being fluidly positionable relative to the support in the third state such that in the third state the body is readily positionable by a user and self supporting such that it will maintain its position when released.

9. The surgical component positioner of claim 8 wherein the tensioning mechanism includes at least one tensioning screw operable to increase friction in the linear adjustment mechanism from little or no friction for free movement to moderate friction for fluid movement to high friction for locking.

10. The surgical component positioner of claim 8 wherein the tensioning mechanism includes at least one tension screw mounted to the body, a spring abutting the screw, and a ball abutting the spring, the ball also abutting the support such that the screw is tightenable to compress the spring and press the ball into engagement with the support such that progressive tightening of the screw progressively restrains motion of the body relative to the support.

11. The surgical component positioner of claim 10 wherein the screw is tightenable to fully compress the spring at one position of screw travel to transmit high engagement forces to lock the linear adjustment mechanism.

12. The surgical component positioner of claim 4 further comprising a base, the linear adjustment mechanism being mounted to the base.

13. The surgical component positioner of claim 12 wherein the base further comprises a spike insertable into a bone adjacent a skeletal joint to support the surgical component positioner adjacent the joint.

14. The surgical component positioner of claim 12 wherein the linear adjustment mechanism is mounted to the base for linear translation relative to the base.

15. The surgical component positioner of claim 14 wherein the linear adjustment mechanism is mounted to the base in sliding dovetail relationship.

16. The surgical component positioner of claim 14 wherein the linear adjustment mechanism is linearly translatable in its mounting to the base in a direction perpendicular to the direction of translation of the body relative to the support.

17. The surgical component positioner of claim 1 further comprising a surgical component mounted to the component link, the surgical component including a tracking element trackable by a surgical navigation system to provide positioning information to a user to guide adjustment of the surgical component positioner.

18. A cut guide positioner for knee surgery comprising:
a body;
a cut guide linked to the body with multiple degrees of rotational freedom relative to the body; and
an adjustable locking mechanism operably connected to the cut guide, the locking mechanism having at least three operation states, a first state in which the cut guide is freely positionable by a user relative to the body in the multiple degrees of rotational freedom, a second state in which the cut guide is fluidly positionable by a user relative to the body in the multiple degrees of freedom such that in the second state the cut guide is readily positionable by a user and is self supporting such that it will maintain its position when released, and a third state in which the cut guide position is locked relative to the body.

19. The cut guide positioner of claim 18 wherein the cut guide is linked to the body by a hemispherical head received in a hemispherical seat for three rotational degrees of freedom, the cut guide positioner further comprising a locking mechanism comprising a plunger engageable with the head, the cam lock being operable to alternatively engage each of three distinct cam faces with the plunger to provide three distinct levels of engagement of the plunger with the head to provide three distinct levels of restraint to motion of the head within the seat.

20. The cut guide positioner of claim 18 further comprising a linear adjustment mechanism including a support engaging the body in linear translating relationship such that the body is linearly translatable relative to the support.

21. The cut guide positioner of claim 20 wherein the linear adjustment mechanism further comprises a tensioning mechanism having a least two operation states, a first state in which the body is readily positionable by a user relative to the support and a second state in which the body position is locked relative to the support.

22. The cut guide positioner of claim 20 further comprising a base, the linear adjustment mechanism being mounted to the base in linear translating relationship relative to the base in a direction perpendicular to the direction of translation of the body relative to the support.

23. The cut guide positioner of claim 18 wherein the cut guide is linked to the body by a hemispherical head received in a hemispherical seat and further wherein the cut guide is configured to cut a tibial bone at a knee joint, the cut guide being rotatable relative to the body to set varus/valgus rotation and the cut guide being rotatable relative to the body to set anterior/posterior slope rotation.

24. The cut guide positioner of claim 18 wherein the cut guide is linked to the body by a hemispherical head received in a hemispherical seat and further wherein the cut guide is configured to cut a femoral bone at a knee joint, the cut guide being rotatable relative to the body to set varus/valgus rotation and the cut guide being rotatable relative to the body to set interior/exterior femoral rotation.

25. The cut guide positioner of claim 18 wherein the cut guide further comprises a tracking element trackable by a surgical navigation system to provide positioning information to a user to guide adjustment of the cut guide positioner.

26. A method for positioning a surgical component adjacent a surgical site, the method comprising:
providing a surgical component positioner having a body, a surgical component linked to the body by a ball joint with multiple degrees of freedom relative to the body, and an adjustable locking mechanism;
adjusting a first angular position of the surgical component relative to the body by angling the ball joint with the locking mechanism in an initial position;
adjusting a second angular position of the surgical component relative to the body by angling the ball joint with the locking mechanism in the initial position; and
moving the locking mechanism to a locked position to lock the first and second angular positions of the surgical component.

27. The method of claim 26 wherein adjusting the first angular position further comprises freely adjusting the first angular position to establish an initial rough position with the locking mechanism in the initial position and then moving the locking mechanism to a fluid motion position and precisely adjusting the first angular position while the locking mechanism provides self supporting restraint to the angular adjustment and wherein adjusting the second angular position further comprises freely adjusting the second angular position to establish an initial rough position with the locking mechanism in the initial position and then moving the locking mechanism to a fluid motion position and precisely adjusting the second angular position while the locking mechanism provides self supporting restraint to the angular adjustment.
